(19) United States
(12) Patent Application Publication
(10) Pub. No.: US 2011/0190889 A1
(43) Pub. Date: Aug. 4, 2011

(54) LORDOTIC INTERBODY DEVICE WITH DIFFERENT SIZES RAILS

(75) Inventors: Keith E. Miller, Germantown, TN (US); Rajesh Ramesh, Memphis, TN (US); Craig M. Squires, Cordova, TN (US)

(73) Assignee: WARSAW ORTHOPEDIC, INC., Warsaw, IN (US)

(21) Appl. No.: 12/696,140

(22) Filed: Jan. 29, 2010

(51) Int. Cl. A61F 2/44

(52) U.S. Cl. 623/17.16

(57) ABSTRACT

A vertebral implant for installation in a disc space is disclosed that includes a body defining a first vertebral support rail and a second vertebral support rail extending along a vertical axis. Each vertebral support rail is separated by a channel running circumferentially around at least a portion of the body along a longitudinal axis of the body. The first vertebral support rail has a first height and the second vertebral support rail has a second height. The first height is smaller than the second height. The height or apex of each vertebral support rail is sized and configured to match the concave nature of the endplates of vertebra of the spine.

### BACKGROUND

The present invention relates generally to treatment of the spinal column, and more particularly relates to an interbody fusion device for placement between adjacent vertebral bodies of the vertebra of a spine to maintain a desired orientation and spacing between the adjacent vertebral bodies.

The normal anatomy of the spinal column presents different alignment and rotational characteristics along three spatial planes. In the coronal (or frontal) plane, the vertebra are normally aligned and present no rotation. In the transverse (or axial) plane, the vertebra are likewise normally aligned and present neutral rotation. In the sagittal plane, the vertebra present a certain degree of rotation and translation which form the physiological curvature of the spine; namely, cervical lordosis, dorsa or thoracic kyphosis, and lumbar lordosis.

Interbody fusion procedures are most commonly performed in the lumbar spine. The lumbar region of the human spine is lordotic in shape. Surgeons often want to restore lordosis when they insert the interbody fusion device. As such, some interbody fusion devices are wedge shaped with the narrow end of the wedge towards the posterior aspect of the intervertebral space. The vertebral endplates of the lumbar spine are typically concave in shape. The interbody fusion device contacts each of these concave endplates. A wedge shaped implant does not provide optimal contact with the concave endplates. Thus, there remains a need for improved interbody fusion devices that are sized and configured to specifically fit the geometry of the concave endplates. The present invention satisfies this need and provides other benefits and advantages in a novel and unobvious manner.

### SUMMARY

According to one aspect a vertebral implant for installation in a disc space is disclosed. The vertebral implant includes a body defining a first vertebral support member and a second vertebral support member. The support members extend along a vertical axis, wherein each vertebral support member is separated by a channel running circumferentially around at least a portion of the body along a longitudinal axis of the body. The first vertebral support member has a first height and the second vertebral support member has a second height. In one form, the first height is smaller than the second height and each height being calculated as a function of inducing a proper orientation of respective vertebra.

In one form, the channel is generally semi-circular in shape and extends inwardly away from the vertebral support members. A slot runs through the body from an upper surface of the body to a lower surface of the body along the vertical axis. A channel in a distal end of the body running through the body along the longitudinal axis to the slot that is sized and configured to receive a bone growth material. Each vertebral support member has a wedge-shaped configuration extending in a plane along the longitudinal axis of the body. An upper surface and lower surface of each vertebral support member includes bone engagement members.

In yet another aspect, a vertebral implant for installation into a disc space is disclosed that includes a body including a first vertebral support member and a second vertebral support member. The vertebral support members are separated by a channel running substantially around a longitudinal axis of the body. Each vertebral support member includes an anterior end that tapers downwardly toward a posterior end. The first vertebral support member has an apex having a larger height than that of the second vertebral support member.

In one form, a posterior height of the second vertebral support member is 65-100% of the height of the first vertebral support member. An anterior height of the second vertebral support member is 65-100% of the height of the first vertebral support member. An apex height of the second vertebral support member is 65-95% of a second apex height of the first vertebral support member. Side walls of the first and second vertebral support members can have a convex shape to facilitate insertion of the interbody implant.

Yet another aspect discloses a method of inserting a vertebral implant into a human spine. The method includes providing a body including a first vertebral support member and a second vertebral support member separated by a channel running substantially around a longitudinal axis of the body. Each vertebral support member includes an anterior end that extends toward a posterior end and is configured to match an arcuate shape of vertebral endplates. The body is implanted in a disc space between two respective vertebra. Once in position, the body is rotated about the longitudinal axis such that the first and second vertebral support members are positioned in connection with endplates of the vertebra. Upon rotation the body orients respective vertebra in a predetermined alignment with respect to one another, which in some forms is a lordotic or kyphotic configuration.

The first and second vertebral support members include a bone engagement portion oriented along the longitudinal axis of the body. Bone growth material is inserted into an internal cavity through a passage such that the bone growth material makes contact with the endplates through a vertical slot running through a central portion the body.

Related features, aspects, embodiments, objects and advantages of the present invention will be apparent from the following description.

### BRIEF DESCRIPTION OF THE DRAWINGS

FIG. 1 is a lateral or side view of a human spine illustrating the curvatures of the human spine.

FIG. 2 is a posterior view of an interbody implant positioned between two respective vertebra of the human spine illustrated in FIG. 1.

FIG. 3 is a lateral or side view of the interbody implant positioned between two respective vertebra of the human spine illustrated in FIG. 1.

FIG. 4 is a front or posterior view of the interbody implant.

FIG. 5 is a lateral or side view of the interbody implant.

FIG. 6 is a top view of the interbody implant.

FIG. 7 is a back or anterior view of the interbody implant.

FIG. 8 is a front view of another representative interbody implant.

FIG. 9 is a lateral or side view of the interbody implant illustrated in FIG. 8.

FIG. 10 is a top view of the interbody implant illustrated in FIG. 8.

FIG. 11 is a lateral or side view of the interbody implant illustrating a lordotic angle created by the interbody implant.

FIG. 12 is a top view of the interbody implant inserted at an oblique angle.

FIG. 13 is lateral view of a illustrative interbody implant.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

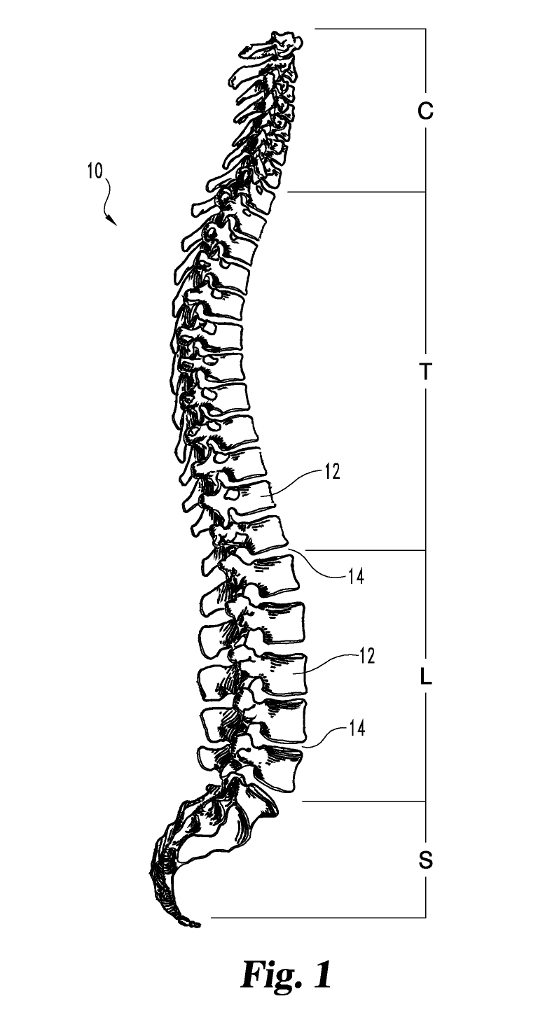

Referring to FIG. 1, a lateral view of a human spinal column 10 is illustrated. As known in the art, the spinal column 10 includes a plurality of vertebra 12 that are stacked vertically on top of one another. The spinal column 10 starts at the base of the skull and continues to the pelvis. Alternate layers of bone (vertebra 12) and cartilage (intervertebral discs 14) stack vertically one on top of the other in the spinal column 10. The intervertebral discs 14 located between the vertebra 12 absorb and distribute shock and keep the vertebra 12 from grinding together during movement. If one of these discs 14 becomes damaged or needs to be removed, the respective vertebra 12 still need to be separated from one another to fill the gap between the vertebra 12 where the disc 14 was once located.

As illustrated in FIG. 1, the spinal column 10 has four natural curves. In particular, the cervical region C of the spinal column 10 is lordotic, the thoracic region T of the spinal column 10 is kyphotic, the lumber region L of the spinal column 10 is lordotic, and the sacral region S of the spinal column 10 is kyphotic. The lordotic regions of the spinal column 10 represent regions that have an increased inward curvature of the spine resulting in a concave back as viewed from the side of the spinal column 10. The kyphotic regions of the spinal column 10 represent regions that have an increased outward curvature of the spine resulting in a convex back as viewed from the side of the spinal column 10.

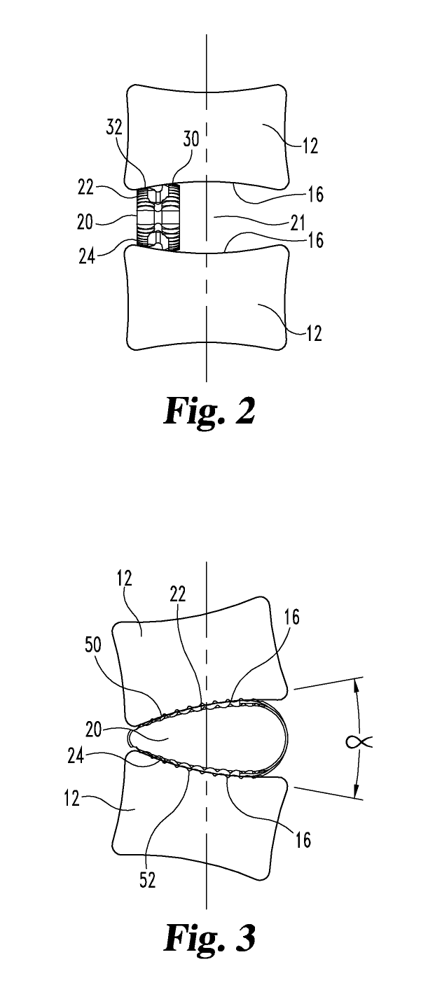

Referring to FIGS. 2 and 3, two vertebra 12 are illustrated with disc 14 removed and an interbody implant 20 has been implanted between the respective vertebra 12 in place of disc 14. In one form, the interbody implant 20 is used to help fuse the two respective vertebra 12 together. The interbody implant 20 is sized and configured to fit between the endplates 16 of the two vertebra 12. As discussed in further detail below, the endplates 16 of each vertebra 12 have a concave shape. In particular, the endplates 16 of the vertebra 12 are hollowed or rounded inward along arcuate paths. The interbody implant 20 is configured to make optimal bone contact with each endplate 16 by increasing the surface area that the interbody implant 20 makes contact with the endplates 16.

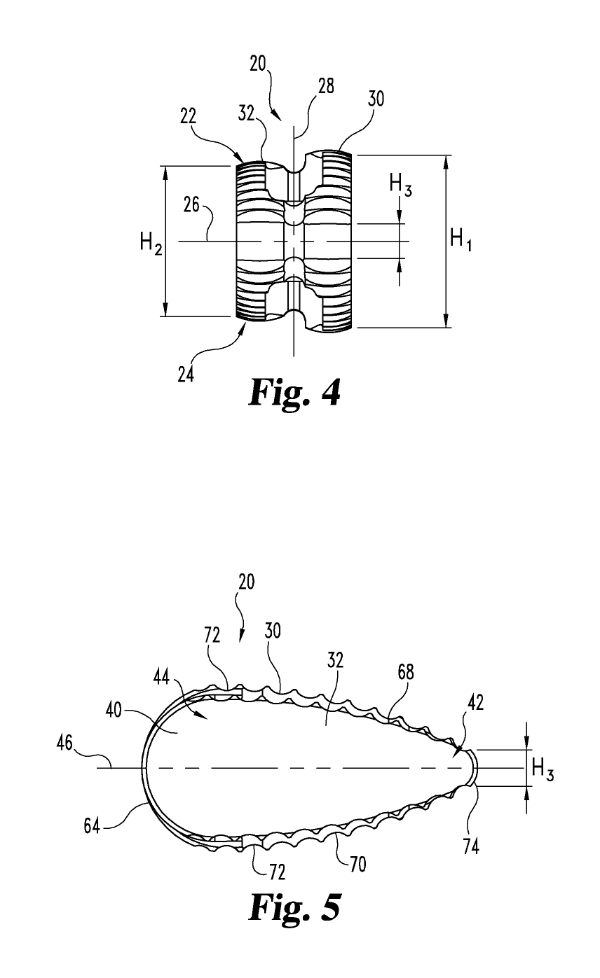

FIG. 2 illustrates a posterior view of the interbody implant 20 positioned in a disc space 21 between two respective vertebra 12. As illustrated, an upper portion 22 of the interbody implant 20 is positioned in a lower endplate 16 of the upper vertebra 12 and a lower portion 24 of the interbody implant 20 is positioned in an upper endplate 16 of the lower vertebra 12. Referring to FIGS. 2 and 4, the interbody implant 20 includes a central lateral axis 26 and a central vertical axis 28. Interbody implant 20 includes a first vertebral support member or medial rail 30 and a second vertebral support member or lateral rail 32. The support rails 30, 32 are shaped in the form of the arcuate curvature of the endplates 16.

The first vertebral support rail 30 extends vertically up and down from lateral axis 26 to a maximum height of H₁. The second vertebral support rail 32 extends vertically up and down from lateral axis 26 to a maximum height of H₂. In one form, the first vertebral support rail 30 has a greater maximum height than the maximum height of the second vertebral support rail 32. As such, in this form H₁ is greater than H₂. Although arcuate or curved rails 30, 32 are illustrated, it is contemplated that straight rails can be used in alternative embodiments. In addition, more than two vertebral support rails can be used in other forms of the present invention.

FIG. 3 illustrates a lateral or side view of the interbody implant 20 positioned between two respective vertebra 12. As illustrated, the upper portion 22 of the interbody implant 20 is positioned in the lower endplate 16 of the upper vertebra 12 and the lower portion 24 of the interbody implant 20 is positioned in the upper endplate 16 of the lower vertebra 12. Referring to FIGS. 3 and 5, in one form the interbody implant 20 has a body 40 that defines the first and second vertebral support rails 30, 32. The body 40 includes a posterior or proximal end portion 42 and an anterior or distal end portion 44 that extend in a plane along the longitudinal axis 46. As previously set forth, the first vertebral support rail 30 has a maximum height or apex of H₁ and the second vertebral support rail 32 has a maximum height or apex of H₂. In this form, the maximum heights H₁, H₂ of the vertebral support rails 30, 32 is located at the distal or anterior end portion 44 of the interbody implant 20.

As the vertebral support rails 30, 32 progress toward the posterior or proximal end portion 42, the heights of the vertebral support rails 30, 32 begin to taper downwardly until reaching the proximal end portion 42 where the vertebral support rails both have a height of H₃. As such, in this form the first vertebral support rail 30 has a maximum height of H₁ at the distal end portion 44 that tapers downwardly to a new height of H₃ at the proximal end portion 42. Likewise, the second vertebral support rail 32 has a maximum height of H₂ at the distal end portion 44 that tapers downwardly to the new height of H₃ at the proximal end portion 42. The vertebral support rails 30, 32 create a wedge-shaped configuration that induces lordotic or kyphotic orientation of the vertebrae 12 when implanted in the disc space 21.

Referring back to FIGS. 2 and 3, as set forth above the endplates 16 of each vertebra 12 have a generally concave or bowl shape. As illustrated, the depth of the endplates 16 changes as you travel from one end of the endplates 16 to the other end with the greatest depth or recess in the endplate 16 occurring at approximately the middle of the endplates 16. As such, interbody implants that include flat upper and lower surfaces that are placed between respective vertebra in contact with the endplates do not provide optimal coverage of the endplate because the endplates have a generally concave or bowl shape.

In this form, since the first vertebral support rail 30 has a greater height than the second vertebral support rail 32, the vertebral support rails 30, 32 provide optimal surface coverage with the endplates 16 of each vertebra 12. As illustrated in FIG. 2, which illustrates a posterior view of the vertebra 12, the difference in height of the first and second support rails 30, 32 allows the support rails 30, 32 to follow the concave curvature of the endplates 16 of each vertebra 12 along the vertical axis 28. In addition, since the endplates 16 have a curved shape along the longitudinal axis 46, the first and second vertebral support rails 30, 32 each have a height at the distal end portion 44 that is sized and configured as a function of the concave curvature of the endplates 16. The vertebral support rails 30, 32 are sized and configured along the longitudinal axis 46 to create maximum surface area coverage of the endplates 16. Different heights (i.e. −H₁, H₂) are used in different locations of the human spine 10 as well as for patients requiring different amounts of space between respective vertebra 12. For example, patients with smaller intervertebral discs 14 that have been removed will require smaller interbody implants 20 having shorter height rails 30, 32 than patients that require greater space between respective vertebra 12.

Referring to FIGS. 3 and 5, which illustrates a lateral view of each vertebra 12 with the interbody implant 20 positioned between the two respective vertebra 12, in this form the interbody implant 20 has a wedge-shape when viewed from the side along the longitudinal axis 46 of the interbody implant 20. In particular, the distal end portion 44 of the first vertebral support rail 30 has a maximum height of H₁ that tapers downwardly along the longitudinal axis 46 toward the proximal end portion 42 where the first vertebral support rail 30 has a shorter height of H₃. Also, the distal end portion 44 of the second vertebral support rail 32 has a maximum height of H₂ that tapers downwardly along the longitudinal axis 46 toward the proximal end portion 42 where the second vertebral support rail 32 has a shorter height of H₃. As such, the interbody implant 20 has a thicker or larger height at the distal end 44 that tapers to a thinner or smaller height at the proximal end 44 thereby taking on the shape of a wedge in this form. However, in alternative forms, the interbody implant 20 can have a teardrop, triangular, oval, egg or generally rectangular shape. In all of these shapes, the interbody implant 20 includes a thicker anterior end that tapers downwardly towards a thinner posterior end.

As further illustrated in FIG. 3, once the interbody implant 20 is properly oriented between the two respective vertebra 12, because of the tapering shape of the first and second vertebral support rails 30, 32, the upper surfaces 50 and lower surfaces 52 of the first and second vertebral support rails 30, 32 make contact with each endplate 16. Referring to FIGS. 3-5, each rail 30, 32 has an arcuate shape in a plane along the longitudinal axis 46 such that the rails 30, 32 fit within the concave endplates 16. Because the rails 30, 32 have an arcuate shape, a substantial portion of the rails 30, 32 make contact with the endplates 16. Further, since the interbody implant 20 is wedge shaped, once the interbody implant 20 is positioned between the respective vertebra 12 the interbody implant 20 induces a lordotic curvature a of the vertebra 12. In other forms, the interbody implant 20 can be positioned such that the interbody implant 20 induces a kyphotic orientation of the two respective vertebra 12. As such, once the fusion process is complete, the vertebra 12 have a lordotic or kyphotic configuration that matches the normal curvature of that particular region of the spine 10.

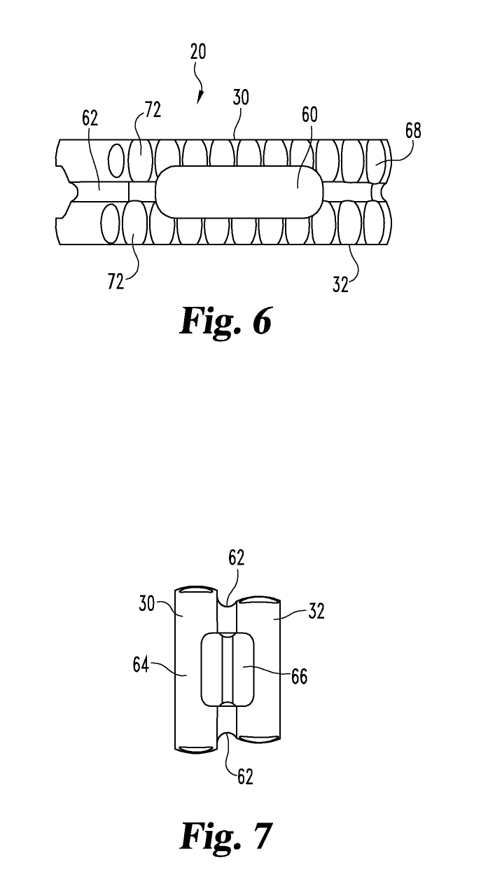

Referring to FIG. 6, the interbody implant 20 includes a slot or void 60 located in a central portion of the interbody implant 20 for the placement of bone growth material. The void 60 runs from the upper surfaces of the interbody implant 20 to the lower surfaces. In particular, slot 60 runs through a portion of rails 30, 32 and a central channel 62. As illustrated, the interbody implant 20 also includes a central channel 62 that runs substantially around the entire body 40 of the interbody implant 20. In one form, the channel 62 has a generally semi-circular shape. The channel 62 is located between the rails 30, 32 and interconnects the rails 30, 32 to one another. In one form, the upper and lower surfaces 68, 70 of the rails 30, 32 are provided with bone engagement members 72, which can be comprised of any one or combination of teeth, grooves, recesses, ridges, knurlings, spikes, or roughened surfaces for securely engaging the endplates 16.

Referring to FIG. 7, the anterior or distal end 64 of the interbody implant 20 includes a generally rectangular channel 66 that extends into the void 60. The channel 64 could also be placed on the posterior end 74 in other representative forms. The channel 66 is sized and configured for bone growth material to be inserted into an internal cavity defined by the channel 66 and the void 60. Any suitable osteogenetic or osteoinductive material or composition is contemplated for placement within the void 60 and channel 66 of any of the implant embodiments discussed herein. Such material includes, for example, autograft, allograft, xenograft, demineralized bone, synthetic and natural bone graft substitutes, such as bioceramics and polymers, and osteoinductive factors. Where bony material is placed within the cavity, the material can be pre-packed into the cavity before the device is implanted. A separate carrier to hold the materials within the cavities of the implants can also be used. These carriers can include collagen-based carriers, bioceramic materials, such as BIOGLASS®, hydroxyapatite and calcium phosphate compositions. The carrier material can be provided in the form of a sponge, a block, folded sheet, putty, paste, graft material or other suitable form. Moreover, the osteogenetic compositions contained within the implants can comprise an effective amount of a bone morphogenetic protein, transforming growth factor .beta.1, insulin-like growth factor 1, platelet-derived growth factor, fibroblast growth factor, LIM mineralization protein (LMP), and combinations thereof or other therapeutic or infection resistant agent, held within a suitable carrier material.

Referring to FIG. 5, although the anterior/posterior ends 64, 74 of the rails 30, 32 are illustrated as having a round configuration, other configurations could be utilized in other forms including sharp or squared off. For an anterior or posterior approach, the profile of the posterior edge 74 is smaller than the anterior edge 64 to induce lordosis. Alternatively, the posterior edge 74 could be larger than the anterior edge 64 to induce kyphosis (if used in the thoracic region of the spine 10). In yet another form, for a lateral approach the anterior edge 64 and posterior edge 74 could be substantially the same size. The difference in the total profile of each rail 30, 32 would induce lordosis. When inserting the interbody implant 20 into the lumbar spine from a posterior approach, the anterior edge 64 of the interbody implant 20 is inserted first.

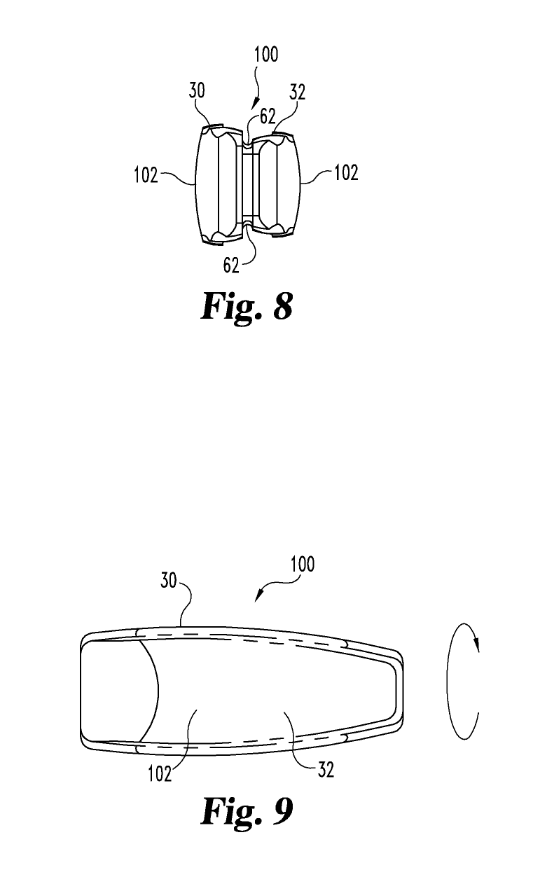

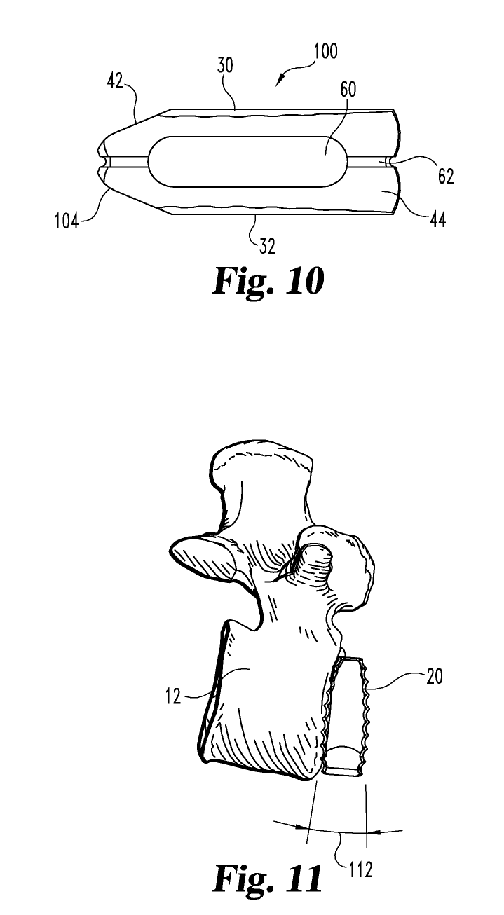

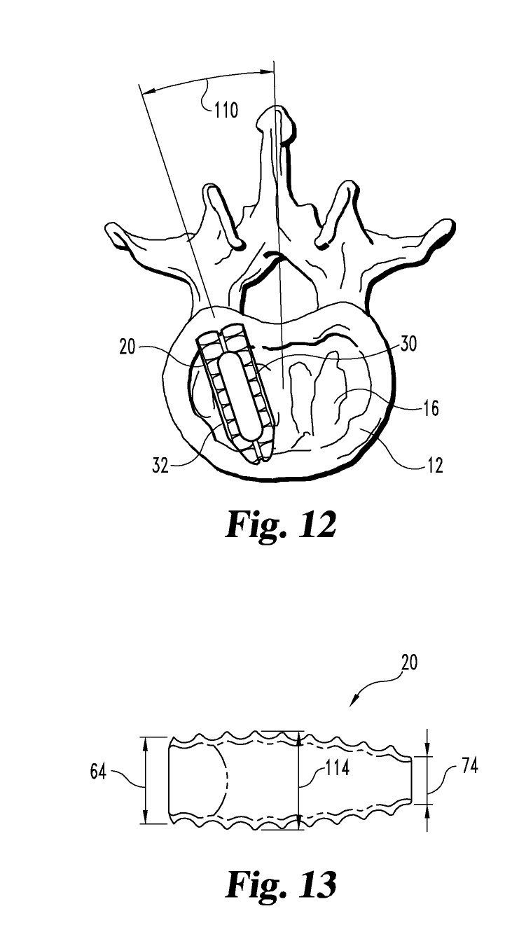

Referring to FIGS. 8-10, another representative interbody implant 100 is illustrated Like numeral references refer to common features of the previously discussed interbody implant 20. In addition, each feature of the interbody implants discussed herein could be incorporated on other forms. In this form, the rails 30, 32 include convex or outwardly curved side walls 102. During implantation, the interbody implant 100 is inserted into the disc space 21 sideways and then rotated 180° so that the rails 30, 32 engage the endplates 16. The convex side walls 102 facilitate rotation of the interbody implant 100 when inserted into the disc space. This is preferred when implanting the interbody implant 100 into the lumbar spine from a posterior approach, because the posterior ligaments do not need to be stretched. The interbody implant 100 also includes a wedge or bullet shaped nose 104 on anterior end portion 42 (see also FIG. 12). The bullet shaped nose 104 also facilitates insertion of the interbody implant 100 into the disc space 21.

Referring to FIGS. 11-13, the interbody implant 20 can also be inserted into the disc space 21 at an oblique insertion angle 110. In this case, the profile or height of each rail 30, 32 is configured to achieve a lordotic angle 112 at the oblique insertion angle 110. In one form, for a posterior approach at the oblique insertion angle 110 (typical TLIF approach), the profile or apex of each rail 30, 32 can be defined as follows: (1) the profile of the posterior end 74 of the lateral rail 32 is 65-100% of the profile of the posterior end 74 of the medial rail 30; (2) the profile of the anterior end 64 of the lateral rail 32 is 65-100% of the profile of the anterior end 64 of the medial rail 30; and (3) the profile of the apex or peak 114 of the lateral rail 32 is 65-100% of the profile of the apex 114 of the medial rail 30.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A vertebral implant for installation in a disc space, comprising:
a body defining a first vertebral support member and a second vertebral support member extending along a vertical axis, wherein each vertebral support member is separated by a channel running circumferentially around at least a portion of said body along a longitudinal axis of said body, wherein said first vertebral support member has a first height and said second vertebral support member has a second height, wherein said first height is smaller than said second height and each height being calculated as a function of inducing a proper orientation of respective vertebra.

2. The vertebral implant of claim 1, wherein said channel is generally semi-circular in shape.

3. The vertebral implant of claim 1, further comprising a slot running through said body from upper surfaces of said body to lower surfaces of said body along said vertical axis.

4. The vertebral implant of claim 3, further comprising a channel in a distal end of said body running through said body along said longitudinal axis to said slot that is sized and configured to receive a bone growth material.

5. The vertebral implant of claim 1, wherein each said vertebral support member has a wedge-shaped configuration extending in a plane along said longitudinal axis of said body.

6. The vertebral implant of claim 1, wherein an upper surface and lower surface of each said vertebral support member includes bone engagement members.

7. A vertebral implant for installation into a disc space, comprising:
a body including a first vertebral support member and a second vertebral support member separated by a channel running substantially around a longitudinal axis of said body, wherein each said vertebral support member includes an anterior end that tapers downwardly toward a posterior end.

8. The vertebral implant of claim 7, wherein said first vertebral support member has an apex having a larger height than said second vertebral support member.

9. The vertebral implant of claim 7, wherein said anterior end is wedged shaped to facilitate insertion into a disc space between two respective vertebra.

10. The vertebral implant of claim 7, wherein said body includes a slot running vertically through a central portion of said body.

11. The vertebral implant of claim 10, wherein said posterior end of said body includes a channel running to said slot.

12. The vertebral implant of claim 7, wherein a posterior height of said second vertebral support member is 65-100% of the height of said first vertebral support member.

13. The vertebral implant of claim 7, wherein an anterior height of said second vertebral support member is 65-100% of the height of said first vertebral support member.

14. The vertebral implant of claim 7, wherein an apex height of said second vertebral support member is 65-95% of the apex height of said first vertebral support member.

15. The vertebral implant of claim 7, wherein side walls of said first and second vertebral support members have a convex shape.

16. A method of inserting a vertebral implant, comprising:
providing a body including a first vertebral support member and a second vertebral support member separated by a channel running substantially around a longitudinal axis of said body, wherein each said vertebral support member includes an anterior end that extends toward a posterior end and is configured to match an arcuate shape of vertebral endplates;
implanting said body in a disc space between two respective vertebra; and
rotating said body about said longitudinal axis such that said first and second vertebral support members are positioned in connection with endplates of said vertebra, wherein upon rotation said body orients respective vertebra in a predetermined alignment with respect to one another.

17. The method of claim 16, wherein said first and second vertebral support members include a bone engagement portion oriented along said longitudinal axis of said body.

18. The method of claim 16, further comprising inserting bone growth material into an internal cavity through a passage such that said bone growth material makes contact with said endplates through a vertical slot running through a central portion said body.

19. The method of claim 16, wherein longitudinal side walls of said first and second vertebral support members have a generally convex shape.

20. The method of claim 16, wherein said anterior end is formed in a wedge shaped configuration to facilitate insertion.
